(19) United States
(12) Patent Application Publication
(10) Pub. No.: US 2016/0228859 A1
(43) Pub. Date: Aug. 11, 2016

(54) USE OF CERTAIN ORGANIC MATERIALS, CONTAINING ALKALI OR ALKALINE-EARTH METALS, FOR IMPLEMENTING ORGANIC CHEMICAL REACTIONS

(71) Applicants: CENTRE NATIONAL DE LA RECHERCHE SCIENTIFIQUE, Paris Cedex 16 (FR); UNIVERSITE MONTPELLIER 2 SCIENCES ET TECHNIQUES, Montpellier Cedex 5 (FR)

(72) Inventor: Claude GRISON, CASTELNAU-LE-LEZ (FR)

(21) Appl. No.: 15/021,070

(22) PCT Filed: Sep. 12, 2014
(86) PCT No.: PCT/FR2014/052280

§ 371 Date: Mar. 10, 2016

(30) Foreign Application Priority Data
Sep. 12, 2013 (FR) 13/58799
Aug. 1, 2014 (FR) 14/57550

(51) Int. Cl. B01J 27/232; B01J 23/02; B01J 37/00; C07C 45/65; B01J 37/34; C07C 51/377; C07C 67/08; C07C 67/297; C07C 1/30; B01J 37/08

(52) U.S. Cl. CPC B01J 27/232; C07C 1/30; B01J 23/02; B01J 37/0036; B01J 37/08; B01J 37/343; C07C 51/377; C07C 67/08; C07C 67/297; C07C 45/65; C07C 2527/232; C07C 2523/02; C07C 2101/14

(57) ABSTRACT

The use of materials of organic origin containing alkali or alkaline-earthmetals, preferably calcium, for the implementation of chemical reactions

The invention relates to the use of materials of organic origin i.e. bio-sourced materials containing alkali or alkaline-earth metals, preferably calcium, for the implementation of chemical reactions.

In international application WO 2011/064462 and application WO 2011/064487 published on 3 Jun. 2011 the invention of Professor Grison and Doctor Escarré is described and claimed, which relates to the use of a calcined plant or a part of a calcined plant having accumulated at least one metal in the M(II) form chosen in particular from zinc (Zn), nickel (Ni) or copper (Cu), for the preparation of a composition containing at least one metal catalyst, the metal of which is one of the aforementioned metals in the M(II) form originating from said plant, said composition being devoid of chlorophyll, and allowing the implementation of organic synthesis reactions involving said catalyst.

Application WO 2011/064487 describes in particular the use of Thlaspi caerulescens which is now called Noccaea caerulescens and Anthyllis vulneraria, as well as numerous other metallophyte plants which are hyperaccumulators of heavy metals for the preparation of catalysts which can be used in organic chemistry.

Therefore the invention described in WO 2011/064487 relates to the use of a calcined plant or part of a calcined plant having accumulated at least one metal in the M(II) form chosen in particular from zinc (Zn), nickel (Ni) or copper (Cu) in which said plant is chosen in particular from the Brassicaceae family, in particular the species of the genus Thlaspi (Noccaea) in particular T. goesingense, T. tatrense, T. rotundifolium, T. praecox, the species of the genus Arabidopsis, in particular Arabidopsis hallerii, and the genus Alyssum, in particular A. bertolonii, A. serpyllifolium, the Fabaceae, the Sapotaceae, in particular the species Sebertia acuminata, Planchonella oxiedra, the Convolvulaceae, in particular the species Ipomea alpina, Planchonella oxiedra, the Rubiaceae, in particular the species Psychotria douarrei, in particular P. costivenia, P. clementis, P. vanhermanii, the Cunoniaceae, in particular the Geissois, the Scrophulariaceae, in particular the species of the genus Bacopa, in particular Bacopa monnieri, algae, in particular red algae, in particular the rhodophytes, more particularly Rhodophyta bostrychia, green algae or brown algae.

Due to this fact, the plant waste is directly recovered and converted to “green” catalysts or to unconventional reagents.

In international patent application WO2013/150197 claiming priority from French patent application FR 2 987 759 filed on 6 Mar. 2012, applications not yet published, Professor Grison and his team have unexpectedly shown that certain other plants which belong to the genus Sedum as well as a different plant, Potentilla griffithi, have metallophyte properties for hyperaccumulating different heavy metals which make them particularly interesting for use in organic chemistry catalysis.

The plants of the genus Sedum are succulents which belong to the Crassulaceae family, composed of more than 400 species. They have the natural aptitude to grow on poor, dry soils, in an open environment and under difficult conditions. Their foliar system is fleshy and they are easy to cultivate.

Among them, three species have developed unusual properties of extracting zinc and cadmium. Sedum plumbizincicola and Sedum jinianum have in particular a remarkable ability to extract zinc from the polluted soils of the south and east of China. They have real potential for phytoextraction and are described as “plumbizincicolafor”.

However, the application of extracts of these plants as catalysts has never been described before and is the subject-matter of French patent application FR 2 987 759 as well as of PCT application No. WO2013/150197.

PCT application No. WO2013/150197 corresponding to French patent application No. FR 2 987 759 also describes the preparation of basic catalysts from accumulating plants indicated above. Different basic catalysts were prepared from the oxides originating from a heat treatment of biomass. The methods described have in common starting from metal oxides, prepared using metal-accumulating plants via drying and heat treatment steps in order to obtain a powder mainly constituted by oxides which after hydration produce the desired basic catalysts which can be supported for example on basic silica or alumina.

Prof Grison's team has thus shown that the plants which hyperaccumulate transition metals such as zinc could be converted to basic catalysts. This property was shown using Sedum plumbinzicola. The results have been incorporated into PCT application No. WO2013/150197 through the isomerization and aldolization reactions of Stetter type allowing access to a 1,4-dione by a green route. This has led to the synthesis of hedione, and more generally to the generalization of the synthesis of cyclopentenones, campholenic aldehyde and derivatives thereof.

In the following, the terms “catalyst” and “reagent” may be used interchangeably. In other words, the alkali or alkaline-earth metals according to the invention and in particular calcium, used preferably in the form of salts or hydroxide can be used as reagents or as catalysts. Unless stated otherwise, the catalysts or reagents used in the implementation of the present invention are basic catalysts.

Prof Grison's team has now studied the activity of basic catalysts or reagents derived from materials of organic origin i.e. from bio-sourced materials and in particular from plants which do not accumulate transition metals i.e. common plants, plants rich in calcium salts, in particular in calcium oxalate and calcifying or non-calcifying algae but rich in calcium as well as reagents derived from marine shells such as the shells of oysters, mussels, scallops, common slipper limpets and other skeletons, carapaces or shells of non-marine molluscs such as snails, all these shells being rich in calcium carbonate.

Examples of such plants are shown in the table below.

| Common plants, in particular plants rich in | Calcifying algae |
|---|---|
| calcium oxalates (families) | (genera) |
| Liliaceae (e.g. dieffenbachia); | green algae: |
| Araceae (e.g. arum maculatum); | e.g. Halimeda |
| Amaryllidaceae (e.g. narcissus); | (aragonite); |
| Chenopodiaceae (e.g. white goosefoot, | Undotea |
| saltbush, quinoa) | brown algae: |
| Lemnaceae (e.g. duckweed); | e.g. Padina |
| Berberidaceae (e.g. mahonia) | red algae: |
| Polygonaceae (e.g. rhubarb, all species of | e.g. Liagoraceae |
| Rumex, the knotweeds) | (Galaxaura/Dichotomaria); |
| Plantaginaceae (e.g. broadleaf plantain) | Corallinaceae: (Amphiroa, |
| Pontederiaceae. (e.g. Pontederia cordata) | Jania) (rhodoliths); |
| Onagraceae (e.g. creeping water primrose); | Lithothamnion (or |
| Portulacaceae (e.g. common purslane) | Breton ameliorant); |
| Agavaceae (e.g. yucca) | Gigartinaceae |
| Moraceae (e.g. iroko) | (e.g. |
| Mosses such as bryophyte-mosses: | Chondrus crispus) |
| Sphagnaceae (e.g. Sphagnum or peat moss) | |
| Fungi such as the polypores: | |
| Inonotus obliquus (or chaga mushroom) | |
| Lichens of the family of the Parmaliaceae | |
| such as Xanthoparmelia conspersa | |

Algae that are non-calcifying but rich in Ca:
Brown algae such as the family of the Alariaceae: for
example Undaria pinnatifida (or wakame),
Alaria esculenta, Alaria marginata, Undaria distans,
Laminaria digitatata, Laminaria saccharina
Red algae such as the family of the Palmariales: for
example Palmaria palmata, family of the Gigartinaceae
example: Chondrus crispus, family of the Bangiophyceae,
Green algae: family of the Ulvaceae, example Ulva lactuca
Marine shells such as the shells of oysters, mussels, scallops,
common slipper limpets skeletons, carapaces and other shells
of non-marine molluscs such as snails, all these shells being
rich in calcium carbonate.

Therefore a subject of the present invention is the use as a catalyst, of materials of organic origin containing alkali or alkaline-earth metals, preferably calcium, for the implementation of organic synthesis reactions involving said basic catalyst.

Therefore a subject of the present invention is also the use as a catalyst, of materials of organic origin containing alkali or alkaline-earth metals, preferably calcium, and practically devoid of transition metals, metalloids and post-transition metals for the implementation of organic synthesis reactions involving said catalyst.

The post-transition metals also called “poor” metals constitute a metallic element of the p-block of the periodic table. This block comprises the following metals: aluminium, gallium, indium, tin, thallium, lead, bismuth, polonium, flerovium.

The term catalyst can also be used in the sense of a reagent. Given the nature of the reactions implemented in the present invention, the two words can be used interchangeably.

By basic catalysis, is preferably meant the use of a catalyst such as the carbonate or the oxide of an alkali or alkaline-earth metal which is preferably calcium. The plants used in the implementation of the invention according to the present application contain calcium in the form of oxalate. Heat treatment of calcium oxalate leads to calcium carbonate or calcium oxide depending on the temperature of the treatment carried out (see for example the preparation of the catalysts A6a and A6b hereafter). As indicated hereafter, the calcifying algae and the shells contain calcium carbonate.

Therefore a subject of the present invention is also the use of extracts of all or part of a member of the kingdom Plantae, preferably a plant or part of a plant, an alga or part of an alga containing a significant level of an alkali or alkaline-earth metal, preferably calcium (Ca), in a quantity preferably greater than 5,000 ppm, more preferentially greater than 50,000 ppm by weight having optionally been subjected to a heat treatment for the preparation of a composition containing at least one basic metal catalyst for the implementation of organic synthesis reactions involving said basic catalyst.

Therefore a subject of the present invention is also the use indicated above characterized in that the materials of organic origin originate from all or part of a member of the kingdom Plantae, preferably a plant or part of a plant, an alga or part of an alga containing a level of an alkali or alkaline-earth metal, preferably calcium (Ca), in a quantity preferably greater than 50,000 ppm by weight, and practically devoid of transition metals, metalloids and post-transition metals, preferably containing less than 20,000 ppm of metals chosen from zinc (Zn), nickel (Ni), manganese (Mn) lead (Pb), cadmium (Cd), copper (Cu) and palladium (Pd), said materials having optionally been subjected to a heat treatment, for the implementation of organic synthesis reactions involving said catalyst.

By member of the kingdom Plantae, is meant plants as well as the lower plants such as algae, fungi, lichens, mosses and ferns.

The alkali metals can be preferably lithium, sodium or potassium, caesium. The alkaline-earth metals are preferably chosen from magnesium, calcium, or barium, more preferentially magnesium or calcium, and yet more preferentially calcium.

The quantity of an alkali or alkaline-earth metal present in the plants which are subjects of the invention is preferably greater than 5,000 ppm, more preferentially greater than 50,000 ppm by weight. In certain cases, this quantity can reach 400,000 ppm.

The levels of metals expressed in ppm are calculated with respect to the total mass of plants having been subjected to dehydration and before optional heat treatment.

The procedure for the optional heat treatment to which the extracts of all or part of a member of the kingdom Plantae, preferably a plant or part of a plant, an alga or part of an alga are optionally subjected is described in the aforementioned international patent application WO 2011/064487 as well as according to the examples provided hereafter. The operation can for example be carried out in a muffle furnace at a temperature of the order of 400° C. to 600° C., preferably of the order of 500° C.

The operation can also be carried out at temperatures of the order of 500° to 600° C. in order to achieve the decomposition of calcium oxalate which is generally of a basicity close to calcium carbonate

The operation can also be carried out at temperatures of the order of 1,000° C. to 1,100° C., in particular when the source of alkali metal, very preferentially calcium (Ca) is a mollusk shell, in order to achieve the decomposition of calcium carbonate to calcium oxide.

A subject of the present invention is also the use after drying and/or grinding and heat and/or chemical treatment of an extract of a plant or part of a plant, an alga or part of an alga comprising a significant level of an alkali or alkaline-earth metal, preferably calcium (Ca), in a quantity preferably greater than 5,000 ppm by weight in the form of salt such as a carbonate or oxalate for the preparation of a composition containing at least one basic metal catalyst preferably in the form of oxide, salt, preferably carbonates, oxalate, or hydroxide of an alkali or alkaline-earth metal, preferably calcium (Ca) for the implementation of organic synthesis reactions involving said basic catalyst.

As indicated above, the invention is implemented with plants rich in calcium. These plants are more particularly rich in calcium oxalate whereas the calcifying algae do not contain calcium oxalate but calcium carbonate.

Calcium salts other than the carbonate and the oxalate can be mentioned, which are present in the aforementioned plants or other plants namely the phosphates such as the phytate, the simple carboxylates such as the citrate, or the osidic carboxylates such as the uronates of alginate or polygalacturonate type.

The drying and/or grinding steps can also be carried out according to the instructions of aforementioned international patent application WO 2011/064487 as well as according to the examples provided hereafter. The grinding can be carried out in demineralized water.

A subject of the present invention is also the use after drying and/or grinding and heat and/or chemical treatment of an extract of a plant or part of a plant comprising a significant level of calcium (Ca), in a quantity preferably greater than 5,000 ppm by weight, in the form of salt such as calcium carbonate or oxalate, for the preparation of a composition containing at least one basic metal catalyst in the form of calcium oxide, a calcium salt chosen from the oxalate, carbonate, phosphates such as the phytate, the simple carboxylates such as the citrate, or the osidic carboxylates such as the uronates of alginate or polygalacturonate type, or calcium hydroxide for the implementation of organic synthesis reactions involving said basic catalyst.

Said compositions contain a low percentage of plant matter in particular after filtration on sintered glass.

A subject of the present invention is also the use of a composition prepared by drying and/or grinding and by heat and/or chemical treatment of an extract of a plant or part of a plant comprising a significant level of calcium (Ca), in a quantity preferably greater than 5,000 ppm by weight, in the form of salt such as calcium carbonate or oxalate and containing at least one basic metal catalyst in the form of calcium oxide, salt such as calcium oxalate or calcium hydroxide, for the implementation of organic synthesis reactions involving said basic catalyst.

A subject of the present invention is also the use as defined above characterized in that the optional chemical treatment(s) to which the extract of a plant or part of a plant is subjected is either a hydration reaction with water, of the oxides obtained by heat treatment or a reaction with a mineral or organic acid preferably hydrochloric acid followed by a treatment with a strong base, preferably soda at a pH greater than 12 of the oxides obtained by heat treatment or of the salts obtained by drying and/or grinding.

A subject of the present invention is also the use as defined above characterized in that the materials of organic origin are extracts of all or part of a member of the kingdom Plantae, preferably a plant or part of a plant, an alga or part of an alga containing a significant level of an alkali or alkaline-earth metal, preferably calcium (Ca), in a quantity preferably greater than 5,000 ppm, more preferentially greater than 50,000 ppm by weight, having optionally been subjected to a heat treatment, for the implementation of organic synthesis reactions involving said basic catalyst.

A subject of the present invention is also the use as defined above characterized in that the materials of organic origin are marine shells or the shells of non-marine molluscs, said shells containing a significant level of an alkali or alkaline-earth metal, preferably calcium (Ca), preferably in the form of calcium carbonate, in a quantity preferably greater than 80%, more preferentially greater than 90% by weight, said shells having optionally been subjected to grinding and/or a heat treatment for the implementation of organic synthesis reactions involving said basic catalyst.

A subject of the present invention is also the use as defined above characterized in that the basic catalyst is an extract of a plant or part of a plant, an alga or part of an alga comprising a significant level of an alkali or alkaline-earth metal, preferably calcium (Ca), in a quantity preferably greater than 50,000 ppm by weight and containing less than 10,000 ppm of metals chosen from zinc (Zn), nickel (Ni), manganese (Mn) lead (Pb), cadmium (Cd), copper (Cu) and palladium (Pd), or of marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight, after drying and/or grinding and/or heat and/or chemical treatment, said catalyst being in the form of calcium oxide, a calcium salt preferably a carbonate, oxalate, or hydroxide of an alkali or alkaline-earth metal, preferably calcium (Ca), for the implementation of organic synthesis reactions involving said basic catalyst.

A subject of the present invention is also the use as defined above characterized in that the basic catalyst is an extract of a plant or part of a plant comprising a significant level of calcium (Ca), in a quantity preferably greater than 50,000 ppm, and containing less than 10,000 ppm of metals chosen from zinc (Zn), nickel (Ni), manganese (Mn) lead (Pb), cadmium (Cd), copper (Cu) and palladium (Pd), or of marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight, optionally after drying and/or grinding and heat and/or chemical treatment said catalyst being in the form of calcium oxide, a calcium salt chosen from the oxalate, the carbonate, the phosphates such as the phytate, the simple carboxylates such as the citrate, or the osidic carboxylates such as the uronates of alginate or polygalacturonate type or calcium hydroxide for the implementation of organic synthesis reactions involving said basic catalyst.

A subject of the present invention is also the use as defined above as a catalyst, of an extract of a plant or part of a plant, of alga or part of an alga comprising a significant level of calcium (Ca), in a quantity preferably greater than 50,000 ppm and containing less than 10,000 ppm by weight of metals chosen from zinc (Zn), nickel (Ni), manganese (Mn) lead (Pb), cadmium (Cd), copper (Cu) and palladium (Pd) prepared by drying and/or grinding and by heat and/or chemical treatment, said catalyst being in the form of salt such as calcium carbonate or oxalate, in the form of calcium oxide or calcium hydroxide, for the implementation of organic synthesis reactions involving said basic catalyst.

By extract is meant a product originating or derived from a plant, an alga or a shell.

A subject of the present invention is also the use as defined above as a catalyst, of marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight, after optionally grinding and/or heat and/or chemical treatment, said catalyst being in the form of calcium oxide, a calcium salt chosen from the oxalate, the carbonate, the simple carboxylates such as the citrate, or the osidic carboxylates such as the uronates of alginate or polygalacturonate type or calcium hydroxide for the implementation of organic synthesis reactions involving said basic catalyst.

A subject of the present invention is also the use as defined above characterized in that the optional chemical treatment(s) to which the extract of a plant or part of a plant, the marine shells or the shells of non-marine molluscs are subjected is either a hydration reaction with water, of the oxides obtained by heat treatment or a reaction with a mineral or organic acid preferably hydrochloric acid followed by a treatment with a strong base, preferably soda at a pH greater than 12 of the oxides obtained by heat treatment or of the salts obtained by drying and/or grinding.

As indicated hereafter, the optional treatment with water is preferably carried out under stirring.

The chemical treatment is preferentially carried out with a mineral or organic acid such as hydrochloric acid, sulphuric acid or acetic acid, preferably hydrochloric acid followed by a treatment with a strong base, preferably soda at a pH greater than 12.

A subject of the present invention is also the use as described above and characterized in that the plant comprising a significant level of an alkali or alkaline-earth metal, preferably calcium (Ca), in a quantity preferably greater than 50,000 ppm by weight, in the form of salt such as calcium carbonate or oxalate is a common plant naturally rich in calcium carbonate or oxalate, preferably calcium oxalate and containing less than 10,000 ppm of metals chosen from zinc (Zn), nickel (Ni), manganese (Mn) lead (Pb), cadmium (Cd), copper (Cu) and palladium (Pd), or an alga chosen from the calcifying algae or the non-calcifying algae, preferably the calcifying algae.

A subject of the present invention is also the use as described above and characterized in that the plant comprising a significant level of calcium (Ca), in a quantity preferably greater than 5,000 ppm by weight, in the form of salt such as calcium oxalate is a plant chosen from:
common plants and in particular plants rich in calcium oxalates chosen from the Liliaceae (such as dieffenbachia); Araceae (such as arum maculatum); the Amaryllidaceae (such as narcissus); the Chenopodiaceae (such as white goosefoot, saltbush, quinoa); the Lemnaceae (such as duckweed); the Berberidaceae (such as mahonia); the Polygonaceae (such as rhubarb, all species of Rumex, the knotweeds); the Plantaginaceae (such as broadleaf plantain); the Pontederiaceae (such as Pontederia cordata); the Onagraceae (such as creeping water primrose); the Portulacaceae (such as common purslane); the Agavaceae (such as yucca); the Moraceae (such as iroko)
calcifying algae chosen from
   the green algae such as Halimeda (aragonite); Udotea
   the brown algae such as Padina
   the red algae such as Liagoraceae (Galaxaura/Dichotomaria);
Corallinaceae: (Amphiroa, Jania) (rhodoliths); Lithothamnion non-calcifying algae chosen from
   the brown algae such as those of the family of the Alariaceae in particular Undaria pinnatifida (or wakame), Alaria esculenta, Alaria marginata, Undaria distans, Laminaria digitata, Laminaria saccharina
   the red algae such as those of the family of the Palmariales in particular Palmaria palmata, those of the family of the Gigartinaceae in particular Chondrus crispus, those of the family of the Bangiophyceae,
   the green algae such as those of the family of the Ulvaceae in particular Ulva lactuca.

A subject of the present invention is also the use as described above and characterized in that the plant comprising a significant level of calcium (Ca), in a quantity preferably greater than 50,000 ppm by weight, in the form of salt such as calcium oxalate or calcium carbonate is a plant chosen from:
the Chenopodiaceae, preferably white goosefoot
the Plantaginaceae, preferably broadleaf plantain
the Portulacaceae, common purslane
the calcifying algae, preferably Lithothamnion.

A subject of the present invention is also the use as described above and characterized in that the plant comprising a significant level of calcium (Ca), in a quantity preferably greater than 50,000 ppm by weight, in the form of salt such as calcium oxalate is a plant chosen from white goosefoot, broadleaf plantain, common purslane or the alga called Lithothamnion.

The shells are preferably chosen from the molluscs, such as the gastropods such as snails, common slipper limpets, the bivalves such as oysters, scallops, mussels, the scaphopods, the polyplacophora and the monoplacophora, the cephalopods, with cuttlefish “bones” or squid “pens”, eggshells, carapaces and skeletons such as those of sea urchins and cnidaria.

The plant that is preferably used can for example be broadleaf plantain, species of Rumex, docks, white goosefoot, the Corallinales, Halimeda aragonite and the alga lithothamnion.

The calcifying algae often have a not inconsiderable level of magnesium.

The non-calcifying algae can also contain a high level of calcium for example of the order of 5,000 to 100,000 ppm.

The shells that are preferably used are the shells of oysters and mussels which are easily available in large quantities.

A subject of the present invention is also the use as described above and characterized in that the materials of organic origin are marine shells such as the shells of oysters, mussels, scallops, common slipper limpets, skeletons, carapaces and other shells of non-marine molluscs such as snails, containing a significant level of an alkali or alkaline-earth metal, preferably calcium (Ca), preferably in the form of calcium carbonate, in a quantity preferably greater than 80%, more preferentially greater than 90% by weight for the implementation of organic synthesis reactions involving said basic catalyst.

A subject of the present invention is also a process for the preparation of a composition as described above and comprising a metallic agent constituted by at least one alkali or alkaline-earth metal, preferably calcium (Ca) characterized in that it comprises the following steps:
a. dehydration, preferably at ambient temperature or in an oven at a temperature of the order of 70° of the biomass comprising the leaves, stems and/or roots of a plant, or an extract of a plant or of an alga or part of an alga comprising a level of an alkali or alkaline-earth metal, preferably calcium (Ca), in a quantity greater than 50,000 ppm by weight in the form of salt such as a carbonate or oxalate and practically devoid of transition metals, metalloids and post-transition metals, preferably containing less than 20,000 ppm of metals chosen from zinc (Zn), nickel (Ni), manganese (Mn) lead (Pb), cadmium (Cd), copper (Cu) and palladium (Pd) and/or
b. Grinding the dry biomass of a plant or an extract of a plant, an alga or part of an alga, optionally obtained after dehydration according to step a)

or
c. Crushing the shells or fragments of shells of animal origin

and/or
d. Heat treatment of the biomass obtained in steps a) or b) in a furnace, preferably in one or more steps preferably in one step at a temperature of the order of 400° C. to 600° C., preferably of the order of 500° C. for several hours, preferably for approximately 7 hours in order to obtain an alkali or alkaline-earth metal salt, preferably calcium carbonate

or
e. Heat treatment of the mass obtained in steps a) or b) or c) in a furnace, preferably in one or more steps, preferably in one step at approximately 1,000° for several hours, preferably for approximately 7 hours, in order to obtain an alkali or alkaline-earth metal oxide, preferably calcium oxide
   and, if desired the salts of an alkali or alkaline-earth metal, preferably calcium (Ca) or the oxides obtained in steps d), and e) respectively are subjected
f. Either, in the case of the oxides obtained in step e) to hydration preferably followed by decantation, evaporation and drying at a temperature of the order of 80° C.
g. Or in the case of the salts, preferably calcium carbonate, obtained in step d) to a treatment with a mineral or organic acid, preferably hydrochloric acid followed by a treatment with a strong base, preferably soda at a pH greater than 12, and if desired

the salts of an alkali or alkaline-earth metal, preferably calcium (Ca), obtained in step d) and the hydroxides of an alkali or alkaline-earth metal, preferably calcium (Ca), obtained in steps f) or g) are mixed, preferably by co-grinding with a support such as alumina, for example basic alumina or hydrotalcite in order to obtain a supported catalyst.

Optional ultrasonic activation is carried out by partial immersion of the reactor in an ultrasonic tank containing a 0.1% aqueous solution of detergent. Sonication is maintained for approximately 15 minutes.

The sintered glass with a porosity of 0-1 (pores of a large size 100 to 250 microns) is placed on a vacuum flask using a rubber cone which makes it possible to ensure sealing. Filtration is carried out under reduced pressure using a water pump. Only the chlorophyll plant residues are retained. However, if the operation is carried out at temperatures of 1,000 to 1,100°, there can be no chlorophyll residues and this step does not need to be carried out.

Different mineral supports can be used for supporting the catalyst and thus producing catalysis on a support. Typically, montmorillonite K10, silica, alumina or hydrotalcite have been used as a support.

The catalysts on mineral supports can preferably be prepared by co-grinding.

The catalysts on mineral supports can be prepared as follows:

The mineral support is introduced into a flask equipped with a magnetic stirrer, then the catalyst preferably of the CAT A1 or CAT A2 type. Water is then added, the resulting suspension is then stirred at ambient temperature for 5 h. This is then filtered, the solid is washed with distilled water, then this is collected for drying in an oven (120° C.) overnight. Once its mass has stabilized, the resulting catalyst is stored in a desiccator.

The catalyst can also be supported on mineral solids, in particular of natural or synthetic origin, such as hydrotalcite. This solid is used as a catalyst support prepared according to the following procedure: the hydrotalcite and the catalyst of the CAT A1 or CAT A2 type are placed in a mortar. The mixture is co-ground at ambient temperature then placed in the oven while waiting to be used.

However, the basic catalysts according to the present invention are preferably used without a support.

A subject of the present invention is also a process as described above for the preparation of a composition comprising a metallic agent constituted by at least one alkali or alkaline-earth metal, preferably calcium (Ca), characterized in that it comprises the following steps:
a) dehydration, preferably at ambient temperature or in an oven at a temperature of the order of 70°, of the biomass comprising the leaves, stems and/or roots of a plant or an extract of a plant or of an alga or part of an alga comprising a level of an alkali or alkaline-earth metal, preferably calcium (Ca), in a quantity greater than 50,000 ppm by weight in the form of salt such as a carbonate or oxalate and practically devoid of transition metals, metalloids and post-transition metals, preferably containing less than 20,000 ppm of metals chosen from zinc (Zn), nickel (Ni), manganese (Mn) lead (Pb), cadmium (Cd), copper (Cu) and palladium (Pd) and/or
b) grinding the biomass, comprising the leaves, stems and/or roots of a plant or part of a plant, an alga or part of an alga, optionally obtained after dehydration according to step a),

and, if desired,
c) ultrasonic activation or gentle heating at a temperature of the order of 60° C. followed by removal of the plant part, preferably on a high-porosity frit, from the ground mixture obtained in steps a) or b) and centrifugation or filtration

and if desired,
d) treatment of the salts such as a carbonate or oxalate of an alkali or alkaline-earth metal, preferably calcium (Ca), obtained in steps a), b) or c) with a mineral or organic acid preferably hydrochloric acid followed by a treatment with a strong base, preferably soda at a pH greater than 12, a treatment preferably followed by centrifugation or filtration of the precipitated solid

and if desired
e) heat treatment of the biomass obtained in step a) or of the ground mixture obtained in step b) in a furnace, preferably in one or more steps, preferably in one step at a temperature ranging from approximately 400° to approximately 1,100° for several hours, preferably for approximately 7 hours
   and, if desired, the oxides of an alkali or alkaline-earth metal, preferably calcium (Ca), obtained in step e) are subjected
f) Either to hydration preferably followed by decantation, evaporation and drying at a temperature of the order of 80° C.
g) Or treated with a mineral or organic acid, preferably hydrochloric acid, followed by a treatment with a strong base, preferably soda at a pH greater than 12, a treatment preferably followed by centrifugation or filtration of the precipitated solid,
   and, if desired,
h) the salts such as a carbonate or oxalate of an alkali or alkaline-earth metal, preferably calcium (Ca), obtained in steps a), b) or c), the oxides of an alkali or alkaline-earth metal, preferably calcium (Ca), obtained in step e) and the hydroxides of an alkali or alkaline-earth metal, preferably calcium (Ca), obtained in steps d), f) or g) are mixed, preferably by co-grinding with a support such as alumina, for example basic alumina, silica, the zeolites, for example montmorillonite, or hydrotalcite, in order to obtain a supported catalyst.

A subject of the present invention is also a process for the preparation of a catalyst comprising a metallic agent constituted by at least one alkali or alkaline-earth metal, preferably calcium (Ca) characterized in that it comprises the following steps:
a) Grinding marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight
   and/or
b) heat treatment of marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight or of the ground biomass obtained in step a), in a furnace preferably in one or more steps, preferably in one step at approximately 1,000 to 1,1000 for several hours, preferably for approximately 7 hours
   and if desired
c) ultrasonic activation or gentle heating at a temperature of the order of 60° C. followed by removal of the plant part, preferably on a high-porosity frit, from the ground mixture obtained in steps a) or b) and centrifugation or filtration and if desired,
d) treatment of the salts such as an oxalate or carbonate of an alkali or alkaline-earth metal, preferably calcium (Ca), obtained in steps a), b) or c) with a mineral or organic acid, preferably hydrochloric acid, followed by a treatment with a strong base, preferably soda at a pH greater than 12, a treatment preferably followed by centrifugation or filtration of the precipitated solid
   and, if desired, the oxides of an alkali or alkaline-earth metal, preferably calcium (Ca), obtained in step b) are subjected
e) Either to hydration, preferably with water, preferably followed by decantation, evaporation and drying at a temperature of the order of 80° C.
f) Or treated with a mineral or organic acid, preferably hydrochloric acid, followed by a treatment with a strong base, preferably soda at a pH greater than 12, a treatment preferably followed by centrifugation or filtration of the precipitated solid
   and, if desired,
g) the salts such as a carbonate of an alkali or alkaline-earth metal, preferably calcium (Ca), obtained in steps a), b) or c), the oxides of an alkali or alkaline-earth metal, preferably calcium (Ca), obtained in step b) and the hydroxides of an alkali or alkaline-earth metal, preferably calcium (Ca), obtained in steps d), f) or g) are mixed, preferably by co-grinding with a support such as alumina, for example basic alumina, silica, the zeolites, for example montmorillonite or hydrotalcite in order to obtain a supported catalyst.

A subject of the present invention is also a process as described above for the preparation of a composition comprising a metallic agent constituted by at least one alkali or alkaline-earth metal, preferably calcium (Ca) characterized in that it comprises the following steps:
a) dehydration, preferably at ambient temperature or in an oven at a temperature of the order of 70° of the biomass comprising the leaves, stems and/or roots of a plant or an extract of a plant or of an alga or part of an alga comprising a significant level of an alkali or alkaline-earth metal, preferably calcium (Ca), in a quantity preferably greater than 50,000 ppm by weight in the form of salt such as a carbonate or oxalate and practically devoid of transition metals, metalloids and post-transition metals, preferably containing less than 20,000 ppm of metals chosen from zinc (Zn), nickel (Ni), manganese (Mn) lead (Pb), cadmium (Cd) copper (Cu) and palladium (Pd)
   and/or
b) Grinding the dry biomass of a plant or an extract of a plant, an alga or part of an alga, optionally obtained after dehydration according to step a)
   and if desired
c) Ultrasonic activation or gentle heating followed by removal of the plant part, preferably on a high porosity frit, from the ground mixture obtained in step a) or b) and centrifugation or filtration
   and, if desired, the salts such as a carbonate or oxalate of an alkali or alkaline-earth metal, preferably calcium (Ca), obtained in steps a), b) or c) are subjected:
d) to a treatment with a mineral or organic acid, preferably hydrochloric acid followed by a treatment with a strong base, preferably soda at a pH greater than 12,
   and, if desired,
e) the salts such as a carbonate or oxalate of an alkali or alkaline-earth metal, preferably calcium (Ca), obtained in steps a), b) or c) and the hydroxides of an alkali or alkaline-earth metal, preferably calcium (Ca), obtained in step d) are mixed, preferably by co-grinding with a support such as alumina, for example basic alumina, the zeolites, for example montmorillonite, silica or hydrotalcite in order to obtain a supported catalyst.

A subject of the present invention is also a process as described above for the preparation of a composition comprising a metallic agent constituted by at least one alkali or alkaline-earth metal, preferably calcium (Ca) from the leaves, stems and/or roots of a plant or an extract of a plant or of an alga or part of an alga comprising a significant level of an alkali or alkaline-earth metal, preferably calcium (Ca), in a quantity preferably greater than 50,000 ppm by weight in the form of salt such as a carbonate or oxalate and practically devoid of transition metals, metalloids and post-transition metals, preferably containing less than 20,000 ppm of metals chosen from zinc (Zn), nickel (Ni), manganese (Mn) lead (Pb) and cadmium (Cd), characterized in that it comprises, before the heat treatment of the mass obtained in steps a) or b), a step of extraction of the chlorophyll using an organic solvent, preferably acetone, ethanol or isopropanol.

A subject of the present invention is also a process as described above characterized in that the plant comprising a significant level of calcium (Ca), in a quantity preferably greater than 5,000 ppm by weight, in the form of salt such as calcium oxalate is a plant chosen from:
common plants and in particular plants rich in calcium oxalates chosen from the Liliaceae (such as dieffenbachia); Araceae (such as arum maculatum); the Amaryllidaceae (such as narcissus); the Chenopodiaceae (such as white goosefoot, saltbush, quinoa); the Lemnaceae (such as duckweed); the Berberidaceae (such as mahonia); the Polygonaceae (such as rhubarb, all species of Rumex, the knotweeds); the Plantaginaceae (such as broadleaf plantain); the Pontederiaceae. (such as Pontederia cordata); the Onagraceae (such as creeping water primrose); the Portulacaceae (such as common purslane); the Agavaceae (such as yucca); the Moraceae (such as iroko)
the calcifying algae chosen from
   the green algae such as Halimeda (aragonite); Udotea
   the brown algae such as Padina
   the red algae such as Liagoraceae (Galaxaura/Dichotomaria); Corallinaceae: (Amphiroa, Jania) (rhodoliths); Lithothamnion.

The dry extracts derived from the aforementioned plants and algae are easily converted to bio-sourced basic catalysts according to different possible protocols.

The process for the preparation of the basic bio-sourced catalysts is carried out preferably according to the following methods:

Case of Plant Species of Type A: Common Plants Rich in Calcium Oxalates

1. The metal carbonates can be obtained from the ashes derived from the aforementioned species, then used directly, supported or unsupported: cat A1;
2. The metal hydroxides can be generated by hydration of the oxides, then used supported or unsupported; (the supports can be basic alumina, silica, the zeolites, hydrotalcite): cat A2;
3. The metal hydroxides can be generated from successive treatments of the ashes: treatment with a mineral or organic acid (HCl or H₂SO₄), taken up in soda at a controlled pH (pH=13), centrifugation and drying at 60° C.: cat A3;
4. If the species of type A are rich in calcium oxalate, the heat treatment is not necessary in order to isolate it. The insolubility of calcium oxalate in water is used to advantage in order to rapidly and easily isolate a white solid which is very rich in Ca.

This solid constituted by oxalate is then subjected to a heat treatment in order to obtain the catalyst constituted by calcium carbonate or oxide depending on the temperature to which the oxalate is subjected: cat A4.

### Case of the Species of Type B: For the Algae

The calcifying algae are dried and finely ground. In this way calcium carbonate is obtained which can be subjected to a heat treatment of the order of 1,000° in order to obtain calcium oxide: cat B.

All of these bio-sourced catalysts are easily available, very easy to use and are excellent substitutes for corrosive bases which are dangerous to use and avoid any reactive treatment. They are removed by simple filtration of the reaction medium and treatment with oxalic acid if necessary. The simplicity of this process contrasts with the conventional use of pure and commercial calcium hydroxide, which is presented as a solid which is difficult to handle in a protic medium. It is then presented as a sticky, viscous product, the texture and the specific surface of which are unsuited to such methods. The polymetallic character of the medium confers on the solid the appearance of a finely divided solid, which can be easily handled and is reactive. It is due to the presence of cations of physiological origin (enzymatic cofactors, magnesium derived from chlorophyll, phosphates etc.) which allow a very good dispersion of the calcium.

### EXPERIMENTAL PART

### Preparation of the Catalysts

### Description of the Different Methods:

CAT A1: 420 g of dehydrated samples of plants rich in Ca are burnt in a muffle furnace at 500° C. for 7 h. 183 g of a solid rich in metal carbonates are obtained.
supported CAT A1: 1.7 g of a solid rich in metal carbonates is co-ground with 5 g of support (e.g. basic alumina, Montmorillonite), then activated by heating for 15 minutes at 150° C.
The solid rich in carbonate also comprises other anions, in particular phosphates.
CAT A2: 10 g of metal carbonates originating from the previous heat treatment is introduced into a beaker and sprinkled with water under stirring. 30 mL of water is added. A grey suspension is obtained; the stirring is maintained for 1 h.
After decantation, the pH is 10. The medium is concentrated in a rotary evaporator, dried in an oven at 80° C. 10.1 g of a grey powder is obtained.
supported CAT A2: 1.7 g of metal carbonates is co-ground with 5 g of support (e.g. basic alumina), then activated by heating for 15 minutes at 150° C.
CAT A3: 10 g of CAT A1 is introduced into a 250 mL flask with 50 mL of 12M HCl added under stirring. The solution obtained is filtered on celite. After evaporation and concentration, 4.9 g of a solid is collected and dried at 80° C. 3 g of the previous solid is dissolved in 70 mL of distilled water with a few drops of HCl in order to promote complete dissolution. Precipitation of the metal hydroxides is carried out by the addition of a concentrated soda solution up to pH=13. The white suspension obtained is centrifuged. 2.7 g of solid is obtained and stored in an oven.
CAT A4: 50 g of leaves are cut up and ground in demineralized water. After ultrasonic activation or gentle heating, the plant part is removed using a high-porosity frit (size 1). The residual aqueous phase is centrifuged. The solid residue, rich in calcium oxalate, is treated as in 3 (heat treatment of plantain at 1,000° C. which leads to a composition close to cat A3).

Typical compositions are presented below according to the origin of the natural species. They were analyzed by ICP MS. The results are expressed in percentages by mass. In the table below and in the following tables, only the content of the metal cations of interest is indicated. The content of anions (in particular oxalates, carbonates or phosphates) is not shown in this table. The sum of the percentages is therefore less than 100%.

The FIG. 13.9 indicated for calcium means that the sample analyzed
contains 13.9 g of calcium per 100 g of total mass.

| | % by mass | | | | | | | | | | |
|---|---|---|---|---|---|---|---|---|---|---|---|
| | Ca | Mg | Zn | Mn | P | S | Se | K | Na | Cu | Fe |
| Cat A3 | 13.9 | 1.6 | 0.4 | 0.3 | 3 | — | — | 21.0 | — | 0.0 | 0.2 |
| Derived from common plants | | | | | | | | | | | |
| e.g. broadleaf plantain | | | | | | | | | | | |
| Cat B | 34.1 | 3.5 | 0.06 | 0.4 | 0.17 | 4.8 | 0.01 | 2 | 3.7 | 0.01 | — |
| Derived from calcifying algae | | | | | | | | | | | |
| e.g. Lithothamnion | | | | | | | | | | | |

These new catalysts were tested in different reactions such as: isomerization, chemoselective hydrolysis of acid-derived functions, controlled esterification, alkylation, elimination, aldolization and similar reactions, carbonyl olefination reactions. The initial hypothesis is confirmed: some of these reactions are possible without a transition metal (e.g. isomerization, chemoselective hydrolysis, controlled esterification, intramolecular and intermolecular aldolizations, alkylation, elimination, carbonyl olefination). These results are very important from an industrial point of view, as they make it possible to extend the field of bio-sourced catalysts to raw materials which are very easily available; finally, the protocols are very simple to implement.

The basic entity can be an oxide, a carbonate or preferably a hydroxide of alkali and particularly alkaline-earth derivatives. It can be supported or unsupported.
CAT A5: the catalyst derived from oyster shells is prepared as follows:

The shells are directly placed in a furnace to be calcined. The furnace is heated at 1,000° C. for approximately 7 h. The resulting powder which is very rich in calcium oxide, is stored under an inert atmosphere, or used directly. In this case, it is rehydrated by progressive additions to water. The pH is then approximately 12. The aqueous phase obtained can constitute the reaction medium or undergo a sequence of filtration/oven drying. In this case, the solid obtained can be used as a catalyst or reagent in another medium, such as an ethanolic medium.

| | % by mass | | | | | | | | | | |
|---|---|---|---|---|---|---|---|---|---|---|---|
| | Ca | Mg | Zn | Mn | P | S | Se | K | Na | Cu | Fe |
| Cat A5 | 51.2 | 0.7 | 0.07 | 0.1 | 0.02 | — | — | 0.08 | 1.09 | 0.0 | 2.3 |
| Derived | | | | | | | | | | | |
| from | | | | | | | | | | | |
| oysters | | | | | | | | | | | |

CAT A6: 95 g of fresh plantain, washed and dried is placed in a 1 L flask containing 500 mL of ethanol. The mixture is taken to reflux for 1 hour under mechanical stirring. The solution is filtered and the residual solid is dried under reduced pressure at 100° C.
90 g of plant matter is treated at 500° C. for 7 h in order to obtain 7.70 g of a residue rich in metal carbonates, called CAT A6a.
If the 90 g of plant matter is treated at 1,000° C. for 7 h, then a solid rich in metal oxides is obtained, called CAT A6b.

Treatment with a green organic solvent (ethanol, propanol, acetone) makes it possible to reduce the concentration of Mg and increase the concentration of Ca and of Ba of the catalyst prepared, and its basicity. It therefore makes it possible to reduce the catalytic mass of the catalyst used. A mineral plantain composition treated (CAT A6b) or not treated (CAT A6c) with an organic solvent, then calcined at 1,000° C. illustrates the modification of mineral composition expressed in ppm.

Mineral composition in ppm of a catalyst (or reagent) of plant origin after being treated or not treated with a green solvent

| | Na | Mg | Al | K | Ca | Cr | Mn | Fe | Ni | Cu | Zn | Cd | Ba | Pb |
|---|---|---|---|---|---|---|---|---|---|---|---|---|---|---|
| CAT | 1674 | 22311 | 2888 | 1711 | 317230 | 43 | 1247 | 3086 | 82 | 265 | 1050 | 67 | 1289 | 197 |
| A6b | | | | | | | | | | | | | | |
| CAT | 4391 | 30927 | 10241 | 3528 | 261767 | 172 | 1715 | 8426 | 494 | 80 | 1180 | 36 | 963 | 227 |
| A6c | | | | | | | | | | | | | | |

CAT A6b and CAT A6c have a reactivity close to CAT A5. The presence of other metallic elements allows easy and non-exothermic hydration.

A subject of the present invention is also the use of a composition as described above containing at least one basic metal catalyst comprising an extract having optionally been subjected to a heat treatment of a plant or part of a plant, an alga or part of an alga comprising a significant level of an alkali or alkaline-earth metal, preferably calcium (Ca), in a quantity preferably greater than 50,000 ppm by weight and practically devoid of transition metals, metalloids and post-transition metals, preferably containing less than 20,000 ppm of metals chosen from zinc (Zn), nickel (Ni), manganese (Mn) lead (Pb) and cadmium (Cd) or of marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight for the implementation of organic synthesis reactions of functional conversions by catalysis involving said basic catalyst characterized in that said organic synthesis reactions are chosen from isomerization, chemoselective hydrolysis, controlled esterification, transesterification, alkylation, elimination reactions, carbon-carbon bond formations such as the aldolization-type, C-glycosidation reactions, carbonyl olefination reactions such as the Wittig-Homer reaction, transesterification, acylation, epimerization, condensation, cyclization, di-, tri-polymerization and addition reactions, conjugate or not conjugate.

A subject of the present invention is also the use of a composition as described above characterized in that the organic synthesis reactions are chosen from hydrolysis reactions and more particularly chemoselective hydrolyses of acid-derived functions, transesterification reactions and intramolecular and intermolecular aldolization reactions or similar.

More precisely, a subject of the present invention is also the use of a composition as described above containing at least one basic metal catalyst comprising an extract which has been optionally subjected to a heat treatment of a plant or part of a plant, an alga or part of an alga comprising a significant level of an alkali or alkaline-earth metal, preferably calcium (Ca), in a quantity preferably greater than 5,000 ppm by weight or of marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight for the implementation of functional conversion organic synthesis reactions by catalysis involving said basic catalyst characterized in that said organic synthesis reactions are chosen from the reactions for the preparation of α,β-ethylenic carbonyl derivatives by isomerization, the cross aldolization reactions using aldehydes that are diversely functionalized such as vanillin or masked in the hemiacetalic form such as the aldoses, the monoesterification of carboxylic diacids, the mild hydrolysis of plurifunctional esters, the transesterification of carboxylic esters.

More precisely, a subject of the present invention is also the use of a composition as described above containing at least one basic metal catalyst for the implementation of organic synthesis reactions involving said basic catalyst, the concentration of alkali or alkaline-earth metal, preferably calcium (Ca), is comprised between 5,000 and 400,000 mg·kg⁻¹ for the calcium content and between 3,000 and 300,000 mg·kg⁻¹ for the magnesium content in the catalysts originating from the leaves, stems and/or roots of a plant or part of a plant or an alga or part of an alga comprising a significant level of an alkali or alkaline-earth metal.

A subject of the present invention is also one of the compositions as obtained by implementation of the process described above.

More precisely a subject of the present invention is also one of the compositions as obtained by implementation of the process described above and containing at least one basic metal catalyst chosen from an alkali or alkaline-earth metal, preferably calcium (Ca), in a quantity greater than 5,000 ppm, preferably in a quantity greater than 50,000 ppm, and in particular greater than 100,000 ppm in the case of calcium (Ca) and in a quantity greater than 3,000 ppm, preferably in a quantity greater than 40,000 ppm, and in particular greater than 80,000 ppm in the case of magnesium (Mg), comprising at least one of said metals in the form of oxide or salt such as calcium carbonate or oxalate or hydroxide.

Generally, the invention is preferentially implemented with a high level of calcium in order to generate a catalyst that is the most basic possible. Other cations are generally not necessary and the invention can be implemented with a low level of transition metals.

In general magnesium produces poorer experimental results than calcium. It is therefore preferable to lower the level of it in the catalysts.

As indicated previously, when very high temporaries of the order of 1,000-1,100° C. are used during the heat treatment step, no chlorophyll remains in the residues obtained. However, the initial presence of chlorophyll can leave magnesium oxide remaining in the product final, which it may be desirable to remove.

This removal can be carried out under the following conditions:

The biomass constituted by the extracts of all or part of a member of the kingdom Plantae, preferably a plant or part of a plant, an alga or part of an alga, can be treated with ethanol or acetone so as to solubilize the chlorophyll solution. The filtrate thus obtained is practically devoid of chlorophyll and can be used for the remainder of the reactions. A reduction of the order of 5 to 10% of the solid mass of catalyst can thus be obtained.

Magnesium is present in very low quantities in oyster shells.

In practice, a very significant advantage of the use of the catalysts or reagents of the invention which are constituted by alkali or alkaline-earth metals, preferentially by salts such as calcium carbonates or hydroxides, is the possibility that at the end of the reaction they are removed from the reactional aqueous phase by converting them to calcium oxalate (by the addition of oxalic acid) which is a salt that is insoluble in water and precipitates.

There is therefore no need to treat the reactional aqueous phase in order to remove the catalyst used.

### II—Synthetic Applications of the Bio-Sourced Basic Catalysts

### Experimental Section: Application of the Catalysts

Isomerization Reaction

An example of isomerization is the migration of a double bond under thermodynamic control. It can be catalyzed quantitatively by a bio-sourced catalyst of the CATA1 type preferably supported on basic alumina, montmorillonite K10 or hydrotalcite.

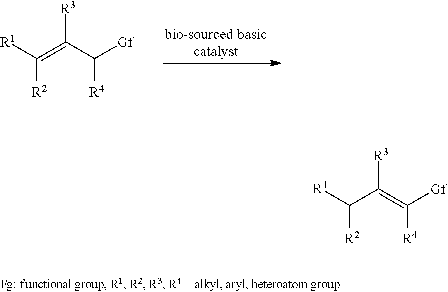

This type of conversion can lead to the preparation of 2-heptenal, a molecule widely used in the perfume and cosmetics industries.

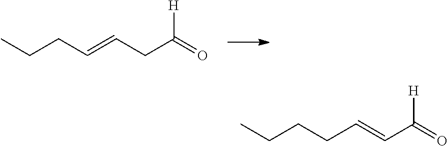

### Experimental Conditions:

50 mg of 3-heptenal is added to a suspension of 2 mL of water and 100 mg of metal oxides (CATA1) supported on basic alumina. The reaction is monitored under IR (vibration band shifts of the C═C double bond from 1659 to 1637 cm-1, and of the C═O double bond from 1726 to 1683 cm-1). After stirring for 1 hour, the reaction is filtered and the medium is evaporated under nitrogen. The reaction is complete (IR and NMR ¹H monitoring).

Chemoselective Hydrolysis of Acid-Derived Functions

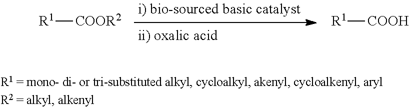

The advantage of the catalysts is to allow mild hydrolyses on fragile or plurifunctional substrates, then to facilitate reaction treatments.

An example is the hydrolysis of an ethyl ester borne by a quaternary carbon: unlike alkali hydroxides, it is possible to avoid the secondary reactions of decarboxylation and isomerization.

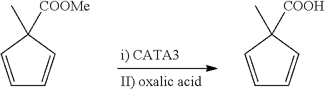

Similarly, the hydrolysis of an ester borne by an aromatic ring, functionalized or not functionalized, is easily carried out using the bio-sourced basic catalysts. The protonation of the carboxylate formed by a natural organic acid, oxalic acid, is doubly advantageous:

it avoids the use of corrosive mineral acids;

it allows the precipitation and recovery of the calcium salts by the formation of insoluble calcium oxalate.

This reaction is preferably carried out with a catalyst derived from oyster shells or a mixture of oyster shells and mussel shells or common slipper limpet shells.

### EXPERIMENTAL CONDITIONS

### Example 1

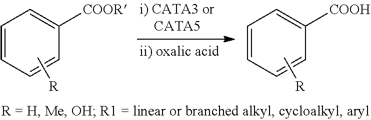

10 mmol of methyl benzoate is added to 100 mg of bio-sourced catalyst preferably of the CATA3 type. A minimum amount of an H₂O/MeOH mixture is added in order to ensure thorough stirring. After heating for 1 h at 60° C., oxalic acid is added. The medium is concentrated. Sublimation of the medium allows very pure benzoic acid to be obtained.

### Example 2

6.327 mL of methyl salicylate (7.403 g, 48.65 mmol, 1 eq.) is added to 125 mL of milliQ water, then the catalyst CAT A5 derived from a mixture of oyster and mussel shells (90% CaO relative to the total mass of the cat., 1 eq. of Ca) is introduced in small portions into a 250 mL three-necked flask under magnetic stirring.

The reaction medium is taken to reflux for 90 minutes (the coloration of the milky medium can pass from a very slightly green tint to yellow or even orange, depending on the batches of catalyst). The pH, initially at 12, reaches the value 9.

Acidification of the reaction medium is achieved with a natural organic acid, oxalic acid.

Three objectives are expected:
Protonation of the phenate
Protonation of the carboxylate
Precipitation of the calcium salts in the form of calcium oxalate.

12.3 g (2 eq.) of oxalic acid dihydrate are added hot until a pH-2 is obtained. The coloration of the very dense milky medium passes to pink then light violet. The precipitation of calcium oxalate is observed. After stirring for 5 minutes, the reaction medium is filtered hot on a frit with a porosity of 4. The pale yellow filtrate is washed with hot water, concentrated in a 250 mL flask, cooled to 5° C. then filtered on the previous frit containing the previous solid residue. The total conversion of the methyl salicylate is confirmed by TLC (eluent: cyclohexane/acetone: 8/2). The salicylic acid is recrystallized from water (solubility in water at 100° C.=66 g/L, at 25° C.=2.24 g/L, at 0° C.=1.2 g/L). The crystals are filtered on a frit with a porosity of 3, rinsed with 20 mL of milliQ water at 50 C and dried under reduced pressure over P₂O₅ for 12 h. 97% white crystals are obtained. The purity of the salicylic acid is verified by GC/MS>98%; characterizations: GC/MS, IR, M.p.: 157-159° C. An ICP/MS analysis confirms the absence of contamination of the salicylic acid with calcium.

Controlled Esterification:

The example of the controlled monoesterification of the primary alcohol of vanillyl alcohol in the presence of the free phenol function is conclusive. The bio-sourced basic catalysts described are much milder and more selective than the conventional acetic anhydride/pyridine system which does not allow this chemoselectivity.

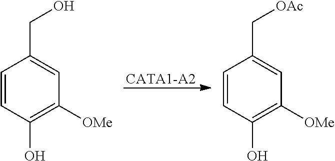

The catalyst CAT A5 is also a transesterification catalyst. Thus, it is capable of facilitating the transesterification of ethyl butyrate by methanol. Supported on a porous silica, the lixiviation phenomena are limited.

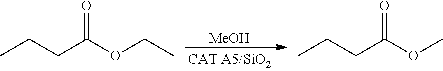

Another reaction of interest is the monoesterification of the natural carboxylic diacids. Monitoring the monoesterification is possible due to the mild basic properties of the catalysts used. The proposed process is based on three successive one-pot conversions:

formation of the anhydride in situ;

trapping the anhydride formed by the alcohol present;

precipitation of the salt derived from the acid ester.

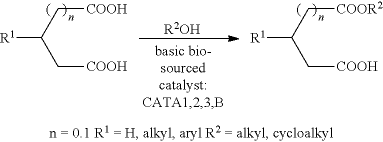

### Example

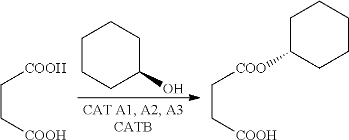

10 mmol of succinic acid is mixed with 100 mg of metal oxides (CATA1). The mixture is heated until sublimation of the succinic anhydride is observed. 10 mmol of cyclohexanol is added to the medium. This is heated at 70° C. The reaction is monitored by TLC (vanillin detection); it is quantitative. No diester formation is observed. The medium is neutralized with oxalic acid.

Metal hydroxides (CAT A 2, A3) and lithothamnion (CATB) can be used; in this case, prior or in situ sublimation of the diacid to anhydride is necessary if it is desired to achieve the same yields.

Alkylation

The bio-sourced basic catalysts are capable of promoting alkylation reactions.

Thus, for example, it is possible to carry out the “O-alkylation” of a carboxylic acid.

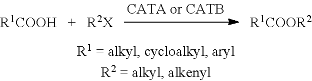

The protocol is exemplified with an alkylating agent such as propyl bromide and acetic acid.

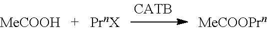

Operating Conditions

Example: 10 mmol of acetic acid and 10 mmol of bromo n-propane are mixed with 100 mg of lithothamnion (CAT B). After 2×30 seconds of microwave activation at 600 W, the mixture is filtered and analyzed by IR. Formation of the propyl acetate is complete (ν (C═O)=1735 cm¹).

Elimination

The 2nd-order beta-eliminations can be catalyzed by the bio-sourced catalysts.

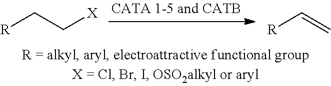

A formula in which alkyl represents a unit having from 1 to 6 carbon atoms, linear or branched, preferably methyl and aryl preferably represents a phenyl radical. An example is the conversion of 2-bromo ethylbenzene to styrene which is easily carried out with all of the catalysts CATA 1-5 and CATB by microwave activation (2 minutes, 400° C., 600 W).

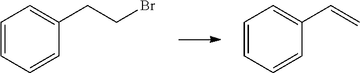

Aldolization and Similar Reactions:

The reactions involving an acidic hydrogen in the alpha position of one or two electroattractive functional groups and an electrophilic partner such as a carbonylated derivative (aldehyde or ketone) or an acid derivative (nitrile, carboxylic ester or anhydride) are promoted by the bio-sourced basic catalysts.

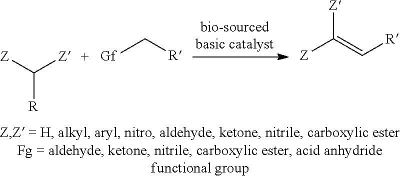

Examples in which Z and/or Z′ are ketone or carboxylic ester groups, and Fg are ketone or aldehyde groups illustrate these possibilities via intra- and inter-molecular pathways. * intramolecular: intramolecular aldolization reactions can be carried out regardless of the catalyst used and the natural species from which it originates. They allow access to key molecules of the cosmetics industry, such as dihydrojasmone.

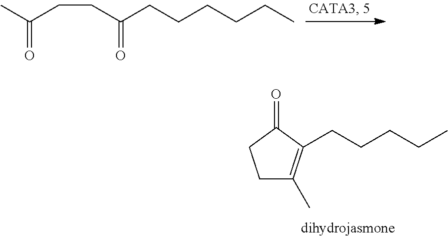

Operating Conditions

2,5-undecadione (0.5 mol) is diluted in a mixture of water and ethanol (80/20 mL). 500 mg of hydroxides from plantain prepared by alkaline hydrolysis of the corresponding chlorides (CATA3) or from CAT A5. The solution is taken to reflux for 16 h, then extracted with ether. Monitoring the reaction by GC MS shows the complete formation of dihydrojasmone.

The catalyst is recycled by filtration, washing with water, with ethanol then acetone and drying for 4 h at 120° C.

* intermolecular: intermolecular aldolization reactions are also possible.

They are illustrated by the Claisen-Schmidt reaction allowing rapid access to styrylketones. These compounds are synthetic intermediates of pyrazonyl derivatives which are of medical interest or are sweeteners. The benefit of the proposed method is to avoid the concurrent Cannizzaro reaction. The quantitative synthesis of chalcone reflects this possibility. All of the proposed catalysts are possible; however CATA3 has the best catalytic activity.

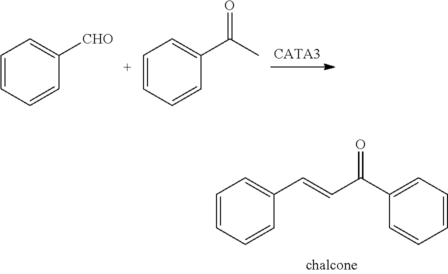

### Operating Conditions:

0.1 mol of benzaldehyde and 0.1 mol of acetophenone are diluted in 20 mL of absolute ethanol. 500 mg of catalyst constituted by a co-ground mixture of 200 mg of the hydroxide from Rumex (CATA3) and basic alumina are added to the reaction medium. After stirring for 2 h under reflux, the reaction medium is cooled down with an iced water bath. The solid precipitate is filtered and recrystallized from ethanol. Extraction of the aqueous phase with hexane makes it possible to obtain a quantitative yield. The product is characterized by its melting point (M.p.°: 57-59° C.), by IR (ν C═O: 1665 cm⁻¹, C═C: 1608 cm⁻¹) and its purity is verified by GC MS.

The crossed aldolization reaction can be extended to aromatic aldehydes and plurifunctional ketones. Examples of industrial interest leading to synthetic fragrances, compounds are used in certain cosmetic or analgesic creams. Their synthesis is carried out from vanillin, corresponding ketones and from the basic catalysts developed. An example is the green synthesis of gingerol, shogaol.

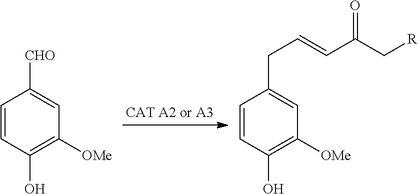

Reactions of the Knoevenagel type are also very efficient thanks to this new type of bio-sourced catalysis. This possibility is demonstrated by the reaction between a β-ketoester and the masked aldehyde function of an aldose. The hemiacetal equilibrium is easily shifted towards the aldehyde form; the adduct undergoes a dehydration reaction in situ. The elimination product is then subjected to an intramolecular Michael reaction.

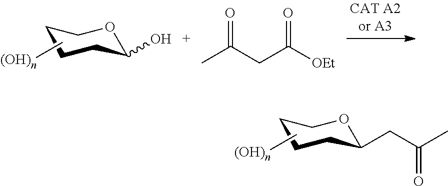

All these take place as one-pot conversions and can be extended to numerous substrates: the β-ketoester can be replaced by a β-dione; the aldose can be a hexose, a pentose, or a deoxyaldose.

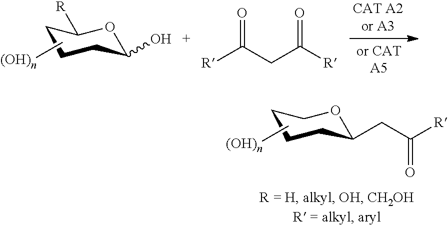

Example: 405 mg of D-xylose is diluted in 15 mL of ethanol, then 1.2 equivalents of pentanedione (332.2 μl) and 326 mg of hydrated CAT A5 derived from Bouzigues oysters (at 80.5% CaO, i.e. 0.75 equivalent of calcium) are added. The reaction mixture is heated for 5 h at 50° C. The calcium acetate formed is filtered and the medium is concentrated. The reaction is quantitative and can be monitored by TLC (DCM/MeOH: 9/1, Rf 0.3). The diastereomeric ratio is evaluated by GC MS after silylation; it is greater than 90/10.

Carbonyl Olefination Reaction

Carbonyl olefination reactions constitute one of the most important methods of creating a C═C double bond. The calcium-rich bio-sourced catalysts constitute very good catalysts for this type of reaction. This result is exemplified by the Wittig-Homer reaction. The reaction is stereoselective: the E olefin is formed preferentially.

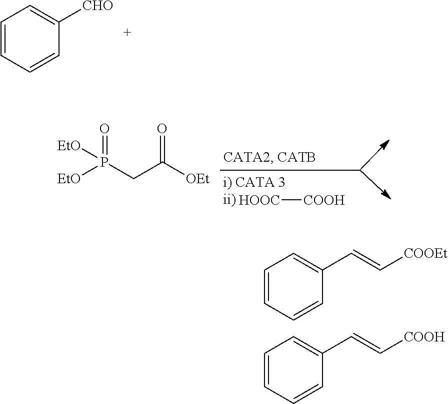

The carbonyl olefination reaction can also be carried out on the hemiacetal function of an aldose. In this case, the carbonyl olefination dehydration-Michael sequence leads to a C-glycoside of interest.

Operating Conditions

10 mmol of ethyl diethyl phosphonoacetate and 10 mmol of benzaldehyde are diluted in a mixture of water and ethanol (10 mL/10 mL). 500 mg of lithothamnion is added to the reaction medium. After stirring for 12 h, 20 mL of hexane is added. After extraction, drying, filtration and evaporation, the organic solution is concentrated under vacuum. >99.9% ethyl cinnamate is obtained.

If the catalyst is an unsupported hydroxide derived from a species rich in calcium oxalate such as Rumex, prolonged stirring allows the hydrolysis of the ethyl cinnamate formed in situ to a cinnamic salt.

1. A method for the implementation of organic synthesis reactions, comprising catalysis involving a catalyst of materials of organic origin containing alkali or alkaline-earth metals, and practically devoid of transition metals, metalloids and post-transition metals.

2. The method according to claim 1, wherein the materials of organic origin originate from all or part of a member of the kingdom Plantae, preferably a plant or part of a plant, an alga or part of an alga containing a level of an alkali or alkaline-earth metal, preferably calcium (Ca), in a quantity preferably greater than 50,000 ppm by weight, and practically devoid of transition metals, metalloids and post-transition metals, preferably containing less than 20,000 ppm of metals chosen from zinc (Zn), nickel (Ni), manganese (Mn) lead (Pb), cadmium (Cd), copper (Cu) and palladium (Pd) and said materials having optionally been subjected to a heat treatment.

3. The method according to claim 1, wherein the materials of organic origin are marine shells or the shells of non-marine molluscs, said shells containing a significant level of an alkali or alkaline-earth metal, preferably calcium (Ca), preferably in the form of calcium carbonate, in a quantity preferably greater than 80%, more preferentially greater than 90% by weight, said shells having optionally been subjected to grinding and/or a heat treatment.

4. The method according to claim 1, wherein said catalyst is a basic catalyst, of an extract of a plant or part of a plant, an alga or part of an alga, comprising a significant level of an alkali or alkaline-earth metal, preferably calcium (Ca), in a quantity preferably greater than 50,000 ppm by weight and containing less than 10,000 ppm of metals chosen from zinc (Zn), nickel (Ni), manganese (Mn) lead (Pb), cadmium (Cd), copper (Cu) and palladium (Pd), or of marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight, after drying and/or grinding and/or heat and/or chemical treatment, said catalyst being in the form of calcium oxide, calcium salt preferably carbonate or oxalate, or the hydroxide of an alkali or alkaline-earth metal, preferably calcium (Ca).

5. The method according to claim 1, wherein said catalyst is a basic catalyst, of an extract of a plant or part of a plant, an alga or part of an alga comprising a significant level of calcium (Ca) in a quantity preferably greater than 50,000 ppm by weight, and containing less than 5,000 ppm of metals chosen from zinc (Zn), nickel (Ni), manganese (Mn) lead (Pb), cadmium (Cd), copper (Cu) and palladium (Pd), or of marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight, optionally after drying and/or grinding and heat and/or chemical treatment said catalyst being in the form of calcium oxide, calcium salt chosen from oxalate and carbonate, the phosphates such as phytate, the simple carboxylates such as citrate, or the osodic carboxylates such as the uronates of alginate or polygalacturonate type or calcium hydroxide.

6. The method according to claim 1, wherein said catalyst is a catalyst, of an extract of a plant or part of a plant, an alga or part of an alga comprising a significant level of calcium (Ca), in a quantity preferably greater than 50,000 ppm and containing less than 5,000 ppm of metals chosen from zinc (Zn), nickel (Ni), manganese (Mn) lead (Pb), cadmium (Cd), copper (Cu) and palladium (Pd) by weight, prepared by drying and/or grinding and by heat and/or chemical treatment, said catalyst being in the form of salt such as calcium carbonate or oxalate, in the form of calcium oxide or calcium hydroxide.

7. The method according to claim 1, wherein said catalyst is a catalyst, of marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight, after optionally grinding and/or heat and/or chemical treatment said catalyst being in the form of calcium oxide, a calcium salt chosen from oxalate and carbonate, the simple carboxylates such as citrate, or the osodic carboxylates such as the uronates of alginate or polygalacturonate type, or calcium hydroxide.

8. The method according to claim 4, wherein optional chemical treatment(s) to which the extract of a plant or part of a plant, the marine shells or the shells of non-marine molluscs are subjected is either a hydration reaction by water of the oxides obtained by heat treatment or a reaction with a mineral or organic acid preferably hydrochloric acid followed by a treatment with a strong base, preferably soda at a pH greater than 12, of the oxides obtained by heat treatment or of the salts obtained by drying and/or grinding.

9. The method according to claim 6, wherein the plant comprising a significant level of an alkali or alkaline-earth metal, preferably calcium (Ca), in a quantity preferably greater than 50,000 ppm by weight, in the form of salt such as calcium carbonate or oxalate, is a common plant naturally rich in calcium carbonate or oxalate, preferably calcium oxalate and containing less than 5,000 ppm of metals chosen from zinc (Zn), nickel (Ni), manganese (Mn) lead (Pb), cadmium (Cd), copper (Cu) and palladium (Pd), or an alga chosen from the calcifying algae or the non-calcifying algae, preferably the calcifying algae.

10. The method according to claim 6, wherein the plant comprising a significant level of calcium (Ca), in a quantity preferably greater than 50,000 ppm by weight, in the form of salt such as calcium oxalate is a plant chosen from:
common plants and in particular plants rich in calcium oxalates chosen from the Liliaceae (such as dieffenbachia); Araceae (such as arum maculatum); the Amaryllidaceae (such as narcissus); the Chenopodiaceae (such as white goosefoot, saltbush, quinoa); the Lemnaceae (such as duckweed); the Berberidaceae (such as mahonia); the Polygonaceae (such asrhubarb, all species of Rumex, the knotweeds); the Plantaginaceae (such as broadleaf plantain); the Pontederiaceae. (such as Pontederia cordata); theOnagraceae (such as creeping water primrose); the Portulacaceae (such as common purslane); the Agavaceae (such as yucca); the Moraceae (such asiroko)
the calcifying algae chosen from
the green algae such as Halimeda (aragonite); Udotea
the brown algae such as Padina
the red algae such as Liagoraceae (Galaxaura/Dichotomaria); Corallinaceae: (Amphiroa, Jania) (rhodoliths); Lithothamnion.
the non-calcifying algae chosen from
the brown algae such as those of the family of the Alariaceae in particular Undaria pinnatifida (or wakame), Alariaesculenta, Alaria marginata, Undaria distans, Laminaria digitata, Laminaria saccharina
the red algae such as those of the family of the Palmariales in particular Palmaria palmata, those of the family of the Gigartinaceae in particular Chondrus crispus, those of the family of the Bangiophyceae,
the green algae such as those of the family of the Ulvaceae in particular Ulva lactuca.

11. The method according to claim 10 wherein the plant comprising a significant level of calcium (Ca), in a quantity preferably greater than 50,000 ppm by weight, in the form of salt such as calcium oxalate or calcium carbonate, is a plant chosen from:
the Chenopodiaceae, preferably white goosefoot
the Plantaginaceae preferably broadleaf plantain
the Portulacaceae common purslane
the calcifying algae preferably Lithothamnion.

12. The method according to claim 11, wherein the plant comprising a significant level of calcium (Ca) in a quantity preferably greater than 50,000 ppm by weight, in the form of salt such as calcium oxalate, is a plant chosen from white goosefoot, broadleaf plantain, common purslane or the alga called Lithothamnion.

13. The method according to claim 1, wherein the materials of organic origin are marine shells such as the shells of oysters, mussels, scallops, common slipper limpets, skeletons, carapaces and other shells of non-marine molluscs such as snails, containing a significant level of an alkali or alkaline-earth metal, preferably calcium (Ca), preferably in the form of calcium carbonate, in a quantity preferably greater than 80%, more preferentially greater than 90% by weight, for the implementation of organic synthesis reactions involving said basic catalyst.

14. Process for the preparation of a catalyst constituted by at least one alkali or alkaline-earth metal, preferably calcium (Ca), characterized in that it comprises the following steps:
a) dehydration, preferably at ambient temperature or in an oven at a temperature of the order of 70°, of the biomass comprising the leaves, stems and/or roots of a plant or an extract of a plant or an alga or part of an alga comprising a level of an alkali or alkaline-earth metal, preferably calcium (Ca), in a quantity greater than 50,000 ppm by weight in the form of salt such as a carbonate or oxalate and practically devoid of transition metals, metalloids and post-transition metals, preferably containing less than 20,000 ppm of metals chosen from zinc (Zn), nickel (Ni), manganese (Mn) lead (Pb), cadmium (Cd), copper (Cu) and palladium (Pd) and/or
b) Grinding the dry biomass of a plant or an extract of a plant, an alga or part of an alga, optionally obtained after dehydration according to step a)
or
c) Crushing the shells or fragments of shells of animal origin
and, or
d) Heat treatment of the biomass obtained in steps a) or b) in a furnace, preferably in one or more steps, preferably in one step at a temperature of the order of 400° C. to 600° C., preferably of the order of 500° C. for several hours, preferably for approximately 7 hours, in order to obtain a salt of an alkali or alkaline-earth metal, preferably calcium carbonate
or
e) Heat treatment of the mass obtained in steps a) or b) or c) in a furnace, preferably in one or more steps, preferably in one step at approximately 1,000° for several hours, preferably for approximately 7 hours, in order to obtain an oxide of an alkali or alkaline-earth metal, preferably calcium oxide
and, if desired, the salts of an alkali or alkaline-earth metal, preferably calcium (Ca) or the oxides obtained in steps d), and e) respectively are subjected
f) Either, in the case of the oxides obtained in step e) to hydration preferably followed by decantation, evaporation and drying at a temperature of the order of 80° C.
g) Or in the case of the salts, preferably the calcium carbonate obtained in step d) to a treatment with a mineral or organic acid, preferably hydrochloric acid, followed by a treatment with a strong base, preferably soda at a pH greater than 12, and if desired
h) the salts of an alkali or alkaline-earth metal, preferably calcium (Ca), obtained in step d) and the hydroxides of an alkali or alkaline-earth metal,
preferably calcium (Ca), obtained in steps f) or g) are mixed, preferably by co-grinding with a support such as alumina, for example basic alumina or hydrotalcite, in order to obtain a supported catalyst.

15. Process for the preparation of a catalyst comprising a metallic agent constituted by at least one alkali or alkaline-earth metal, preferably calcium (Ca) characterized in that it comprises the following steps:
a) dehydration, preferably at ambient temperature or in an oven at a temperature of the order of 70°, of the biomass comprising the leaves, stems and/or roots of a plant or an extract of a plant or of an alga or part of an alga comprising a level of an alkali or alkaline-earth metal, preferably calcium (Ca), in a quantity greater than 50,000 ppm by weight in the form of salt such as a carbonate or oxalate and practically devoid of transition metals, metalloids and post-transition metals, preferably containing less than 20,000 ppm of metals chosen from zinc (Zn), nickel (Ni), manganese (Mn) lead (Pb), cadmium (Cd), copper (Cu) and palladium (Pd) and/or
b) grinding the biomass, comprising the leaves, stems and/or roots of a plant or part of a plant, an alga or part of an alga, optionally obtained after dehydration according to step a),
and, if desired,
c) ultrasonic activation or gentle heating at a temperature of the order of 60° C. followed by removal of the plant part, preferably on a high porosity frit, from the ground mixture obtained in steps a) or b) and centrifugation or filtration
and if desired,
d) treatment of the salts such as a carbonate or oxalate of an alkali or alkaline-earth metal, preferably calcium (Ca), obtained in steps a), b) or c) with a mineral or organic acid preferably hydrochloric acid followed by a treatment with a strong base, preferably soda at a pH greater than 12, a treatment preferably followed by centrifugation or filtration of the precipitated solid
and if desired
e) heat treatment of the biomass obtained in step a) or of the ground mixture obtained in step b) in a furnace, preferably in one or more steps, preferably in one step at a temperature ranging from approximately 400° to approximately 1,100° for several hours, preferably for approximately 7 hours
and, if desired, the oxides of an alkali or alkaline-earth metal, preferably calcium (Ca), obtained in step e) are subjected
f) Either to hydration preferably followed by decantation, evaporation and drying at a temperature of the order of 80° C.
g) Or treated with a mineral or organic acid, preferably hydrochloric acid, followed by a treatment with a strong base, preferably soda at a pH greater than 12, a treatment preferably followed by centrifugation or filtration of the precipitated solid,
and, if desired,
h) the salts such as a carbonate or oxalate of an alkali or alkaline-earth metal, preferably calcium (Ca), obtained in steps a), b) or c) the oxides of an alkali or alkaline-earth metal, preferably calcium (Ca), obtained in step e) and the hydroxides of an alkali or alkaline-earth metal, preferably calcium (Ca), obtained in steps d), f) or g) are mixed, preferably by co-grinding with a support such as for example basic alumina, silica, the zeolites, for example montmorillonite or hydrotalcite, in order to obtain a supported catalyst.

16. Process for the preparation of a catalyst comprising a metallic agent constituted by at least one alkali or alkaline-earth metal, preferably calcium (Ca), characterized in that it comprises the following steps:
a) Grinding marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight
and/or
b) heat treatment of marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight, or of the ground biomass obtained in step a), in a furnace, preferably in one or more steps, preferably in one step at approximately 1,000 to 1,100° for several hours, preferably for approximately 7 hours
and if desired
c) ultrasonic activation or gentle heating at a temperature of the order of 60° C. followed by removal of the plant part, preferably on a high porosity frit, from the ground mixture obtained in steps a) or b) and centrifugation or filtration
and if desired,
d) treatment of the salts such as an oxalate or carbonate of an alkali or alkaline-earth metal, preferably calcium (Ca), obtained in steps a), b) or c) with a mineral or organic acid, preferably hydrochloric acid, followed by a treatment with a strong base, preferably soda, at a pH greater than 12, a treatment preferably followed by centrifugation or filtration of the precipitated solid
and, if desired, the oxides of an alkali or alkaline-earth metal, preferably calcium (Ca), obtained in step b) are subjected
e) Either to hydration preferably followed by decantation, evaporation and drying at a temperature of the order of 80° C.
f) Or treated with a mineral or organic acid, preferably hydrochloric acid, followed by a treatment with a strong base, preferably soda at a pH greater than 12, a treatment preferably followed by centrifugation or filtration of the precipitated solid
and, if desired,
g) the salts such as a carbonate of an alkali or alkaline-earth metal, preferably calcium (Ca), obtained in steps a), b) or c) the oxides of an alkali or alkaline-earth metal, preferably calcium (Ca), obtained in step b) and the hydroxides of an alkali or alkaline-earth metal, preferably calcium (Ca), obtained in steps d), f) or g) are mixed, preferably by co-grinding with a support such as alumina, for example basic alumina, silica, the zeolites, for example montmorillonite or hydrotalcite, in order to obtain a supported catalyst.

17. Process according to claim 14 for the preparation of a composition comprising a metallic agent constituted by at least one alkali or alkaline-earth metal, preferably calcium (Ca), characterized in that it comprises the following steps:
dehydration, preferably at ambient temperature or in an oven at a temperature of the order of 70°, of the biomass comprising the leaves, stems and/or roots of a plant or extract, of a plant or of an alga or part of an alga comprising a significant level of an alkali or alkaline-earth metal, preferably calcium (Ca), in a quantity preferably greater than 50,000 ppm by weight in the form of salt such as a carbonate or oxalate and practically devoid of transition metals, metalloids and post-transition metals, preferably containing less than 20,000 ppm of metals chosen from zinc (Zn), nickel (Ni), manganese (Mn) lead (Pb), cadmium (Cd) copper (Cu) and palladium (Pd)
and/or
b) Grinding the dry biomass of a plant or an extract of a plant, an alga or part of an alga, optionally obtained after dehydration according to step a)
and if desired
b) Ultrasonic activation or gentle heating followed by removal of the plant part, preferably on a high porosity frit, from the ground mixture obtained in step a) or b) and centrifugation or filtration and, if desired, the salts such as a carbonate or oxalate of an alkali or alkaline-earth metal, preferably calcium (Ca), obtained in steps a), b) or c) are subjected:
c) to a treatment with a mineral or organic acid, preferably hydrochloric acid, followed by a treatment with a strong base, preferably soda at a pH greater than 12,
and, if desired,
d) the salts such as a carbonate or oxalate of an alkali or alkaline-earth metal, preferably calcium (Ca), obtained in steps a), b) or c) and the hydroxides of an alkali or alkaline-earth metal, preferably calcium (Ca), obtained in step d) are mixed, preferably by co-grinding with a support such as alumina for example basic alumina, the zeolites for example montmorillonite, silica or hydrotalcite in order to obtain a supported catalyst.

18. Process according to claim 14 for the preparation of a composition comprising a metallic agent constituted by at least one alkali or alkaline-earth metal, preferably calcium (Ca) from the leaves, stems and/or roots of a plant or an extract of a plant or of an alga or part of an alga comprising a significant level of an alkali or alkaline-earth metal, preferably calcium (Ca), in a quantity preferably greater than 50,000 ppm by weight in the form of salt such as a carbonate or oxalate and practically devoid of transition metals, metalloids and post-transition metals, preferably containing less than 20,000 ppm of metals chosen from zinc (Zn), nickel (Ni), manganese (Mn) lead (Pb), cadmium (Cd) characterized in that it comprises, before the heat treatment of the mass obtained in steps a) or b) a step of extraction of the chlorophyll using an organic solvent, preferably acetone, ethanol or isopropanol.

19. Process according to claim 14, wherein the plant comprising a significant level of calcium (Ca), in a quantity preferably greater than 5,000 ppm by weight, in the form of salt such as calcium oxalate is a plant chosen from:
common plants and in particular plants rich in calcium oxalates chosen from the Liliaceae (such as dieffenbachia); Araceae (such as arum maculatum); the Amaryllidaceae (such as narcissus); the Chenopodiaceae (such as white goosefoot, saltbush, quinoa); the Lemnaceae (such as duckweed); the Berberidaceae (such as mahonia); the Polygonaceae (such asrhubarb, all species of Rumex, the knotweeds); the Plantaginaceae (such as broadleaf plantain); the Pontederiaceae (such as Pontederia cordata); theOnagraceae (such as creeping water primrose); the Portulacaceae (such as common purslane); the Agavaceae (such as yucca); the Moraceae (such asiroko)
the calcifying algae chosen from
the green algae such as Halimeda (aragonite); Udotea
the brown algae such as Padina
the red algae such as Liagoraceae (Galaxaura/Dichotomaria); Corallinaceae: (Amphiroa, Jania) (rhodoliths); Lithothamnion.

20. The method according to claim 1, wherein the implementation of organic synthesis reactions is a functional conversion, and the catalysis involves a composition, said at least one basic metal catalyst comprising an extract having optionally been subjected to a heat treatment of a plant or part of a plant, of an alga or part of an alga comprising a significant level of an alkali or alkaline-earth metal, preferably calcium (Ca), in a quantity preferably greater than 50,000 ppm by weight and practically devoid of transition metals, metalloids and post-transition metals, preferably containing less than 20,000 ppm of metals chosen from zinc (Zn), nickel (Ni), manganese (Mn) lead (Pb), cadmium (Cd) or of marine shells or shells of non-marine molluscs containing a significant level of an alkali or alkaline-earth metal, preferably calcium (Ca), preferably in the form of calcium carbonate, in a quantity preferably greater than 80%, more preferentially greater than 90% by weight, and wherein said organic synthesis reactions are chosen from isomerization, chemoselective hydrolysis, controlled esterification, transesterification, alkylation, elimination reactions, carbon-carbon bond formations such as the aldolization-type, C-glycosidationreactions, or carbonylolefination reactions such as the Wittig-Horner reaction, transesterification, acylation, epimerization, condensation, cyclization, di-,tri-polymerization and addition reactions, conjugate or not conjugate.

21. The method according to claim 20, wherein said organic synthesis reactions are chosen from hydrolysis reactions and more particularly chemoselective hydrolyses of acid-derived functions, transesterification reactions and intramolecular and intermolecular aldolization or similar reactions.

22. The method according to claim 20 of wherein said organic synthesis reactions are chosen from the reactions for the preparation of α,β-ethylenic carbonyl derivatives by isomerization, the cross aldolization reactions using diversely functionalized aldehydes such as vanillin or masked in the hemiacetalic form such as the aldoses, the monoesterification of carboxylic diacids, the mild hydrolysis of plurifunctional esters and the transesterification of carboxylic esters.

23. The method according to claim 21, wherein the concentration of alkali or alkaline-earth metal, preferably calcium (Ca) is comprised between 50,000 and 200,000 mg·kg⁻¹ in the case of the calcium content and between 3,000 and 200,000 mg·kg⁻¹ in the case of the magnesium content in the catalysts originating from the leaves, stems and/or roots of a plant or part of a plant, or an alga or part of an alga, comprising a significant level of an alkali or alkaline-earth metal.
